(19) United States
(12) Patent Application Publication
(10) Pub. No.: US 2004/0024319 A1
(43) Pub. Date: Feb. 5, 2004

(54) Clamp for medical use comprising two articulated jaws

(76) Inventor: Bernard Flipo, Nice (FR)

Correspondence Address: YOUNG & THOMPSON, 745 SOUTH 23RD STREET 2ND FLOOR, ARLINGTON, VA, 22202

(21) Appl. No.: 10/332,139

(22) PCT Filed: Jul. 2, 2001
(86) PCT No.: PCT/FR01/02107

(30) Foreign Application Priority Data
Jul. 5, 2000 (FR) 00/008859

(51) Int. Cl. A61B 8/14

(52) U.S. Cl. 600/459

(57) ABSTRACT

The present invention relates to a clamp for medical use comprising two jaws articulated by a pivot and controlled by the practitioner from a gripping region.

According to the invention, one (7) of the jaws (7, 8) comprises pointing means in the tissues of the patient projecting outwardly of the clamp; one (7) of the jaws (7, 8) has two cutting blades (10) on its periphery to section the tissues; at least one of the jaws (7, 8) comprises a cavity (13) for the reception of the cut tissues.

The present invention relates to a clamp for medical se comprising two jaws articulated by a pivot and controlled by the practitioner from a gripping region.

The invention finds its particular application in the industry of production and use of medical instruments particularly for gynecology to take biopsies.

At present, biopsy clamps such as those called “Douai clamps” have two articulated jaws. One of the jaws is bordered by cutting blades to cut tissues. The other comprises the counterpoint of the cutting jaw. The assembly forms, when the jaws are together, a closed volume for the reception of the cut tissues.

Most often, this type of claim is in the form of a device with two legs provided with rings for gripping by the practitioner.

Moreover, a straight intermediate portion ensures the transmission of the control of the jaws from the legs with rings by a system of a sliding rod or cable.

Such jaws have different drawbacks.

In the first place, the jaws are complex and costly particularly as to the mode of actuation.

These claims are generally for multiple use and accordingly require sterilization.

They are costly to buy and represent an important investment for the practitioner to the extent that, in addition to the cost of buying the clamps, it is necessary to add that of installing sterilization and its use.

Another drawback of present clamps is that their use is sometimes sensitive because there are noted Frequent difficulties in the positioning of the clamp in the region from which the removal is to be carried out.

Thus, it is noted that the clamp has a tendency to deviate or slide on the tissue particularly when the jaws are actuated to carry out the removal .

There is known from. DE-U-9005519 a clamp provided with teeth serving not to position the instrument on the tissues, but to grip the removed tissues. This clamp thus provides no solution to the problem of positioning the tool.

There is also known from U.S. Pat. No. 4,597,385 a clamp provided with two juxtaposed teeth at the distal end of a jaw. These teeth are provided to stabilize the tissues during use. Given that, the formation of two spaced teeth on a jaw blocks the possibility for the practitioner to pivot, even slightly, the clamp.

Moreover, the teeth do not project outwardly of the clamp, which renders pointing ineffective.

Moreover, the cutting portion is of a small size and no means is provided for receiving what is removed.

The present invention permits overcoming the drawbacks of the present clamps and provides for this purpose a new clamp for medical use.

A first advantage of this clamp is to provide pointing means permitting, by anchoring in the tissues of the patient, avoiding any risk of deviation or sliding.

There results a greater precision in the removal that is carried out as well as greater safety.

Moreover, the clamp according to the invention is more practical to use than those previously known.

Furthermore, the present clamp can, in a particular embodiment, eliminate the rod that actuates the jaws. In this way, the cost of production of the clamp is substantially decreased and its fabrication from plastic material can be envisaged so as to be disposable.

There are thus avoided all the drawbacks connected with the multiple use of clamps and particularly the cost and time of sterilization.

Other objects and advantages will become apparent from the description which follows which is however given only by way of indication and not for the purpose of limiting the invention.

The present invention relates to a clamp for medical use comprising two jaws articulated by a pivot and controlled by the practitioner from a gripping region, characterized by the fact that one of the jaws comprises means for pointing into the tissues of the patient, projecting outwardly of the clamp.

This clamp can be present in embodiments introduced hereafter:

the pointing means are constituted by a point located at the distal end of one of the jaws and projecting outwardly of the clamp and toward the other jaw.

the pointing means are constituted by two points each located at the distal end of one of the jaws and projecting outwardly of the clamp and toward the other jaw, the two points juxtaposing when the jaws are together.

one of the jaws has two cutting blades on its periphery for sectioning the tissues.

at least one of the jaws comprises a cavity for the reception of the cut tissues.

the cutting blades meet at the distal end of the jaw which carries them, forming two sides of an isosceles triangle.

the blades have a length of about 3 cm and the base of the triangle is of a length of about 2 cm.

the gripping region is formed by two legs with rings connected by a pivot of the jaws, said jaws being each formed in prolongation of one leg, on the side of the pivot opposite the gripping region.

the forward end of the legs, located on the side of the pivot opposite the gripping region, is incurved convexly and is of a material that is resiliently deformable during pressure of the jaws.

the curvature of the two front portions is symmetrical.

the rear portion of the legs, located on the same side of the pivot as the gripping region, is incurved concavely and is of a material resiliently deformable upon pressure of the jaws.

the curvature of the two rear portions is symmetrical.

the distance between the distal end of the jaws and the pivot is 3 to 5 cm.

It comprises:

two jaws connected by a pivot and each having at its rear end a ring to constitute the gripping region;

two secondary legs connected by a pivot comprising the pivot for the jaws, said jaws being each formed in prolongation of the front end of a secondary leg;

articulation means for the front ends of the legs each with the rear end of one of the secondary legs.

the articulation means comprise:

two symmetrical shells each secured to the end of a secondary leg and each provided with a hollow recess extending into the shell along the arc of a substantially perpendicular circle and centered relative to the axis passing through the two pivots, said hollow recesses being provided with an opening oriented opposite the secondary leg;

two hubs each secured to the end of a leg and disposed in the hollow recess of the shells, the hubs being stopped in translation in their hollow recess.

the articulation means comprise:

two symmetrical shells each secured to the end of a leg and provided each with a hollow recess extending into the shell along the arc of a circle substantially perpendicular and centered relative to the axis passing through the two pivots, said hollow recesses being provided with an opening oriented opposite the leg;

two hubs each secured to the end of a secondary leg and disposed in the hollow recess of the shells, the hubs being stopped in translation in their hollow recess.

the hubs are spherical,

the hollow recesses comprise cavities of shapes and dimensions complementary to those of the hub.

the jaw having the cutting blades comprises two recesses in which the blades are inserted.

the clamp is of plastic material.

The accompanying drawings are given by way of indicative examples and are not limiting. They represent a preferred embodiment according to the invention. They permit easy comprehension of the invention.

FIG. 1 is an overall view of the clamp according to the invention in a preferred embodiment.

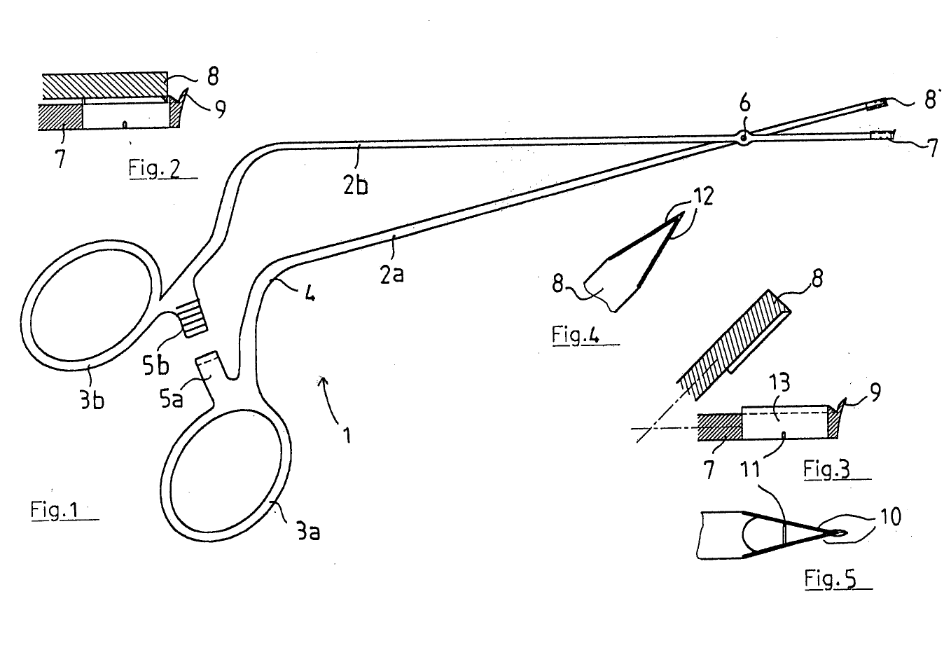

FIG. 2 is a fragmentary cross-sectional view of the jaw in the closed position.

FIG. 3 is a fragmentary cross-sectional view of the jaw portion in the spaced position.

FIG. 4 is a fragmentary view from below of the upper jaw.

FIG. 5 is a fragmentary view from above of the lower jaw.

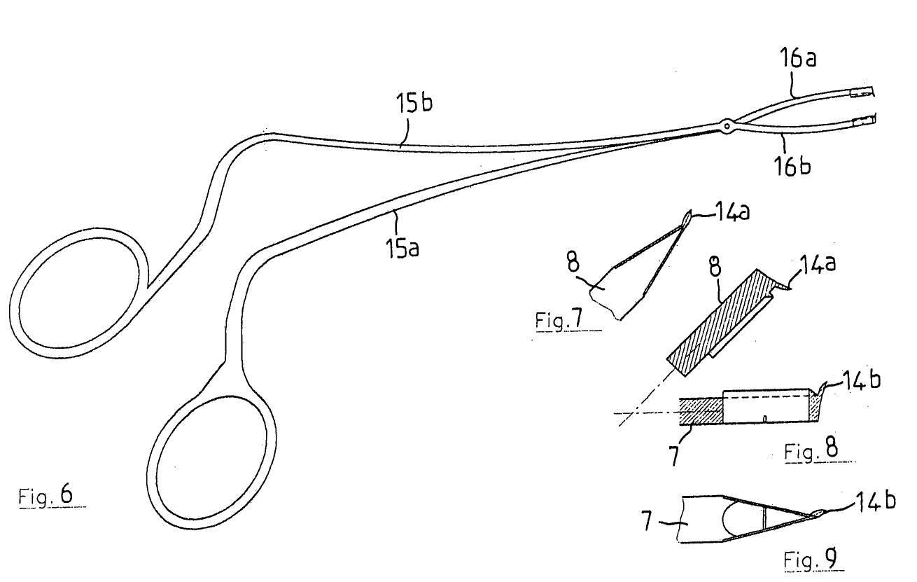

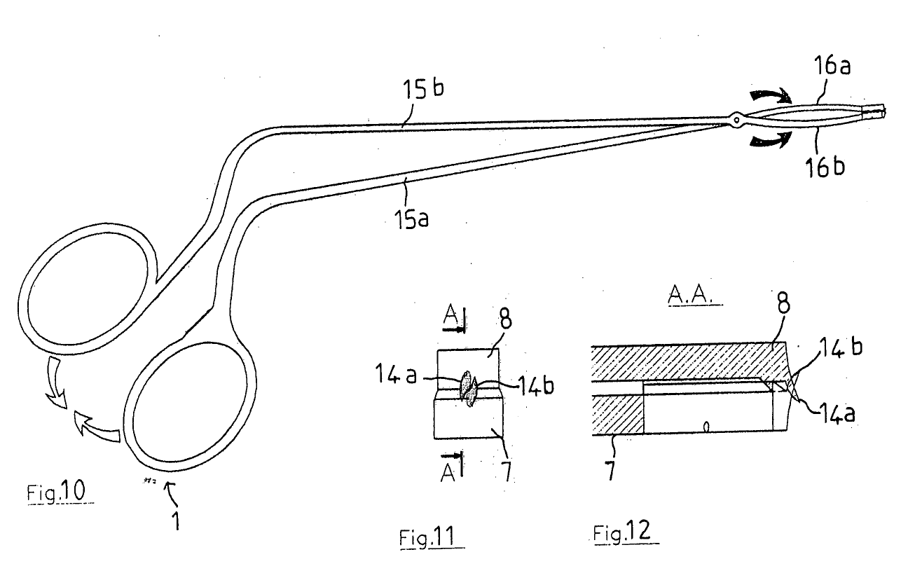

FIG. 6 shows a modified shape of the legs of the clamp; FIG. 10 is another possibility.

FIGS. 7 to 9 show a particular embodiment of the jaws. FIGS. 11 and 12 show its closed position.

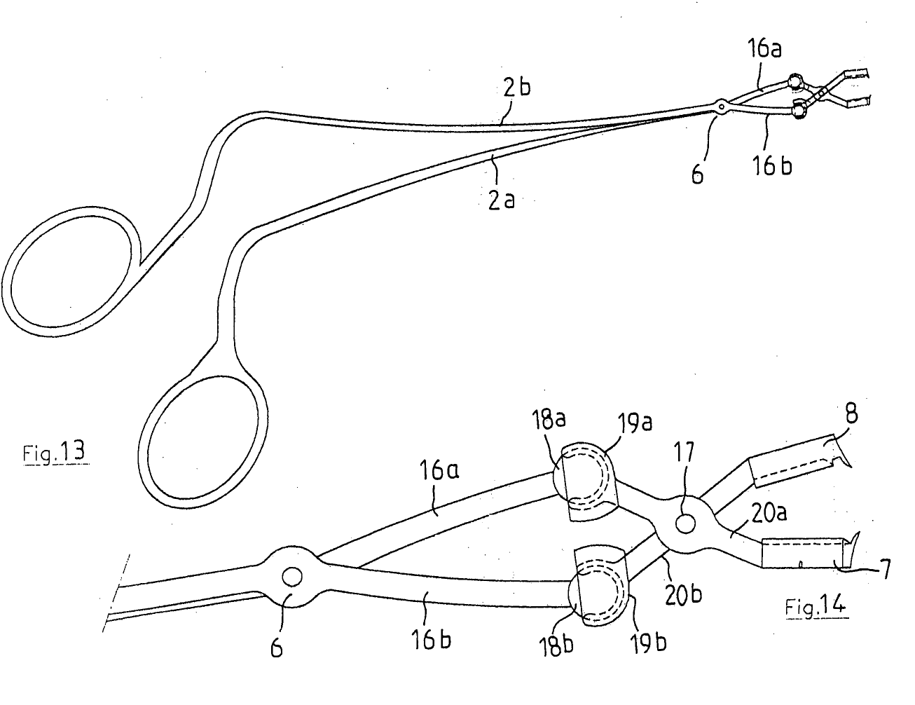

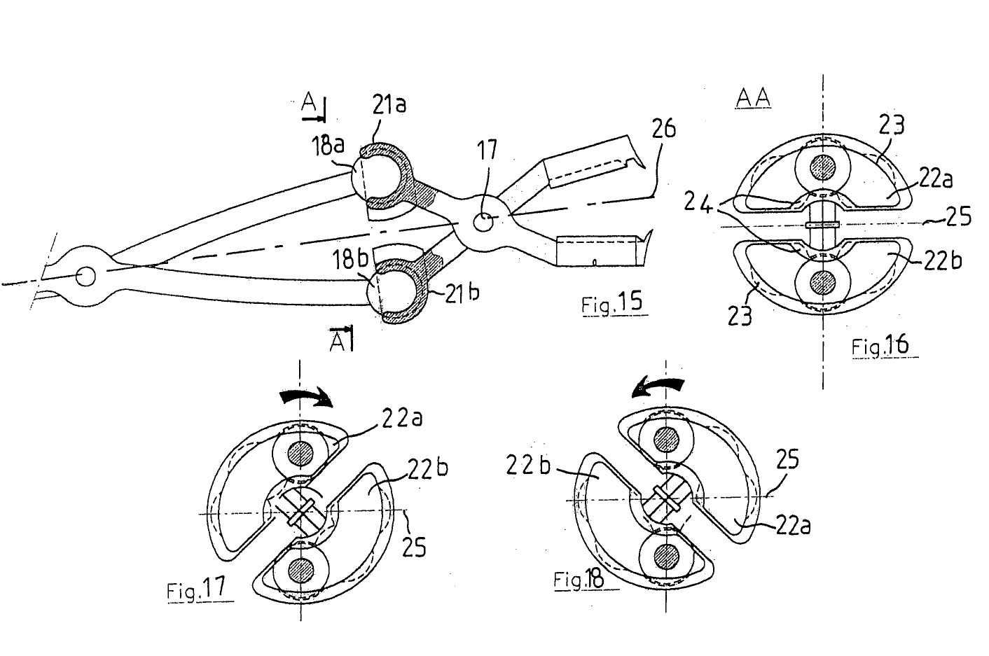

FIGS. 13 to 18 show another embodiment of the invention with a supplemental degree of liberty.

FIG. 1 gives an example of the shape that the clamp could have according to the invention although this example is indicative and does not exclude other configurations of embodiment of the clamp.

According to the example of FIG. 1, the clamp comprises two jaws indicated at 7 and 8, articulated by a pivot so as to be able to move together to a closed position shown in FIG. 2 or spaced as is the case of FIG. 3.

The approach or spacing of the jaws 7, 8 is controlled by a practitioner from a gripping region 1.

In the illustrated embodiment, this gripping region 1 comprises two rings 3a, 3b formed at the end of two legs 2a, 2b.

If desired, the legs 2a, 2b, include an elbow 4. Moreover, closure tongues 5a, 5b can be constituted in a known manner so as to permit holding the clamp in the closed position. The use of such closure tongues is however of secondary importance.

In the case of the example of FIG. 1, the pivotal articulation existing between the two jaws 7, 8 is provided by means of a pivot 6 as is the case for a simple pair of scissors.

In this case, the jaws 7 and 8 are located in prolongation of the legs 2a, 2b, but on the other side of the pivot 6 relative to the gripping region 1.

The jaws 7, 8 can be constituted of a single piece.,. of the material of the legs 2a, 2b, or be pieces added at their end.

According to the invention, one of the jaws 7, 8 comprises means for pointing into the tissues of the patient.

Thus, it has been noted that an important need exists in the provision of an anchor of the clamp in the course of the work of the practitioner.

The advantage of such an anchoring possibility is realized particularly during removal of tissues or in biopsies.

In a preferred embodiment shown in FIGS. 2 and 3, the pointing means take the form of a point 9 located at the distal end of one of the jaws 7.

Referring to FIG. 3, it will be seen that the orientation of the point can be particular.

Thus, it is advantageous to constitute the point 9 oriented not only outwardly by projecting beyond the distal end of the jaw 7, but also toward the other jaw 8.

According to this arrangement, the practitioner can easily hold the clamp in position, by exerting a slight pressure by the point 9 on the tissue and by maintaining this force slightly oriented upwardly.

It will also be noted that this arrangement permits sufficient penetration into the tissue without at the same tire sinking deeply into the latter.

FIGS. 7 to 9 and 11 and 12 show the formation of two points 14a, 14b, each on one jaw 7, 8. In this way, the practitioner can select the jaw 7, 8 which he desires to point into the tissue, which is advantageous for flexibility of positioning of the clamp. The points 14a, 14b are each oriented to one side of the jaw to permit their juxtaposition during closure.

The jaw here shown is particularly applicable to the performance of a biopsy.

In this regard, one of the jaws, the jaw 7, has two cutting blades 10 so as to section the tissues.

As shown in FIG. 5, the blades 10 are present adjacent the edges of the jaw 7.

In the preferred embodiment shown in FIG. 5, the jaw 7 as well as the jaw 8 has a substantially triangular shape at its end.

In this connection, the cutting blades at the distal end of the jaw thus form an isosceles triangle.

By way of example, the blades could each have a length of 3 centimeters, whilst the base of the triangle thus formed will have a length of about 2 centimeters.

Referring to FIGS. 3 to 5, it will be understood that it is possible to section the tissues by means of cutting blades 10 by their coaction with the edges 12 present in a coacting manner on the jaw 8.

Again within the preferred field of performing a biopsy, the jaw can have a cavity 13 to receive the removed tissue.

As shown in FIGS. 3 and 5 for example, the cavity 13 can be delimited in its lower portion of the jaw 7 by a through configuration comprised by one or several bars 11.

FIG. 2 shows that, when the jaws 7 and 8 are together, the cavity formed in the jaw 7 permits providing an interstitial space that is substantially closed.

In the case of the formation of a clamp comprised by two legs 2a, 2bconnected by a pivot 6, the distance. between the distal end 7, 8 and the pivot can be 3 to 5 centimeters.

According to a preferred modification of the invention, the present clamp is adapted to be disposable.

In this connection, it is advantageous to make it essentially of plastic material, particularly of polycarbonate or polyamide.

To do this, most of the constituent elements of the clamp will be constituted of plastic material and the blades 10 could be, either formed integrally, or added and formed of any material presently used, particularly metal, in the form of razor blades.

For the securement and holding in position of the blades 10 on the jaw 7, this latter can comprise recesses (not shown in the drawings) but extending substantially to the position where it is desired to insert the cutting blades 10.

The recesses will each comprise a slot extending along the edges of the jaw 7 and receiving the blades 10.

There is thus provided a clamp whose manipulation and use are practical because they provided pointing means and permit production at least cost so as to constitute a disposable member.

According to a preferred modification, the clamp comprises incurved legs to exert a supplemental force on the jaws, after they have come into contact, by resilient deformation of the legs.

Particularly for an embodiment in plastic material, it is important to preserve the optimum force application characteristics, when the jaws (7, 8) are closed.

To do this, it is proposed preferably to give an incurved shape to the legs (2a, 2b), permitting resilient deformation of the legs when the jaws are in contact, to increase the applied force.

There will be used a material whose properties of resilient deformation are in accord with the desired deformation for the legs (2a, 2b).

The curvature given to the legs (2a, 2b) is the reverse of that which they have a tendency to adopt when force is applied by the laws (7, 8).

Referring to FIG. 10, the end portions (16a, 16b) of the legs (2a, 2b) (these parts are those located on the side opposite the gripping region (1)) having a convex curvature, preferably symmetrically.

The black arrows in FIG. 10 show the direction of resilient deformation which tends to cancel the convex curvature.

In a supplemental or alternative manner, the rear portions (15a, 15b) of the legs (2a, 2b) (these parts are on the same side as the gripping region (1)) have a concave curvature, as shown in FIG. 6.

The production of such a clamp with curved legs as in FIGS. 6 and 10 is possible moreover with any type of design of the jaws.

There will be described hereafter another modification of the invention with reference to FIGS. 14 to 18.

According to this embodiment, it is possible to provide a relative pivot between the portion of the clamp comprising the jaws and the portion of the clamp comprising the gripping region 1.

This additional freedom in rotation permits adjusting optimally the orientation of the jaws when removal of tissues or any other operation is carried out.

It will be noted that the provision of this supplemental degree of freedom is usable for a clamp with any type of jaws.

Referring to FIGS. 13 and 14, there is provided a gripping region as well as legs 2a, 2b substantially equivalent to those described in the preceding embodiment.

Given that, the end portions 16a, 16b of the legs 2a, 2b no longer in this case carry the jaws 7, 8.

The jaws 7, 8 are thus carried by secondary legs 20a, 20b connected pivotally by a pivot 17.

The degree of supplemental rotation is rendered possible by the connection between the legs 2a, 2b and the secondary legs 20a, 20b.

This connection takes place more precisely between the end portions 16a and 16b of the legs 2a, 2b and the rear portions of the secondary legs 20a, 20b.

It will be immediately apparent that the opening and closing of the jaws 7, 8 is carried out by the rings 3a, 3b in the gripping region but this time by means of two rotations about the pivots 6 and 17.

The supplemental articulation provided by the legs 2a, 2b and 20a, 20b ensures adjustment of the orientation of the jaws 7, 8.

According to the embodiment shown in the drawings, these articulation means are substantially symmetrical for respectively the legs 2a, 20a and 2b, 20b

For each of these pairs of legs, there is provided a shell 19a, 19b which in the case shown in the figures is secured to the rear end of the secondary legs 20a, 20b.

The shells 19a, 19b comprise an opening oriented opposite the. legs 20a, 20b and giving access to a hollow recess 22a, 22b formed in the internal volume of the shells 19a, 19b

There is shown more particularly in FIGS. 16 to 18 the shape given to the shells 19a, 19b.

Thus, to carry out a supplemental movement of rotation, the hollow recess 22a, 22b has the shape substantially of an arc of a circle disposed in a plane perpendicular to the axis 26 common to the two pivots 16 and 17, this arc of a circle being moreover centered on this axis 26.

It will also be noted in the drawings that the shape of the shells 19a, 19b and particularly their hollow recess 22a, 22b, is symmetrical.

Each recess 22a, 22b receives a hub 18a, 18b secured to the corresponding end of a leg 2a, 2b.

The hub 18a, 18b is movable in rotation in the recess 22a, 22b but is stopped in translation and held within the hollow recess 22a by any known means or preferably by a suitable size of the opening of the recesses 22a, 22b sufficiently less than the size of the hubs 18a, 18b, so as to retain them.

It will be easily understood that it is possible to reverse the arrangement of the shells 19a, 19b in the hubs 18a, 18b, and that as a result the shells 19a, 19b can be provided at the ends of the legs 2a, 2b and the hubs 18a, 18b can be provided on the ends of the secondary legs 20a, 20b.

Again with reference to FIGS. 13 to 18, the hubs 18a, 18b are substantially spherical and coact with cavities 23, 24 formed within the hollow recesses 22a, 22b.

In this way, the hubs 18a, 18b have the ability to be disposed in one of the cavities 23, 24 and are displaceable only with a slight supplemental effort along the arc of a circle defined by the hollow recesses 22a, 22b.

There is thus formed, depending on the number and arrangement of the cavities 23, 24, a plurality of stop positions of the hubs 18a, 18b in the shells 19a, 19b.

The practitioner thus has a choice between a multiplicity of relative positions of the gripping region 1 and the jaws 7, 8.

Finally, it is pointed out that for all the explanation given above, the term rear is used for any portion of the clamp according to the invention disposed to the rear of the clamp, which is to say toward the gripping region 1.

Conversely, there is called the front any portion of a member constituting the clamp, disposed to the front of the clamp, which is to say toward the jaws 7, 8.

### REFERENCES

1. Gripping region

2a, 2b. Legs

3a, 3b. Rings

4. Elbow

5a, 5b. Closure tongues

6. Pivot

7. Lower jaw

8. Upper jaw

9. Point

10. Blades

11. Bar

12. Edges

13. Cavity

14a, 14b. Points

15a, 15b. Rear portions

16a, 16b. Front portions

17. Secondary pivot

18a, 18b. Hubs

19a, 19b. Shells

20a, 20b. Secondary legs

21a, 21b. Envelope

22a, 22b. Hollow recesses

23. Cavities

24. Cavities

25. Transverse axis of the pivot 6

26. Common axis of the pivots 6, 17

1. Clamp for medical use comprising two jaws (7, 8) articulated about a pivot and controlled by the practitioner from a gripping region (1), characterized. by the fact
that one (7) of the jaws (7, 8) comprises means for pointing in the tissues of the patient projecting outwardly from the clamp;
that one (7) of the jaws (7, 8) has two cutting blades (10) on its periphery to section tissues;
that at least one of the jaws (7, 8) comprises a cavity (13) for the reception of sectioned tissues.

2. Clamp for medical use according to claim 1, characterized by the fact
that the pointing means are constituted by a point (9) located at the distal end of one (7) of the jaws (7, 8) and projecting outwardly of the clamp and toward the other jaw (8).

3. Clamp for medical use according to claim 1, characterized by the fact
that the pointing means are constituted by two points (14a, 14b) each located at the distal end of one of the jaws (7, 8) and projecting outwardly of the clamp and toward the other jaw, the two points (14a, 14b) juxtaposing when the jaws (7, 8) are together.

4. Clamp for medical use according to any one of claims 1 to 3, characterized by the fact
that the cutting blades (10) come together at the distal end of the jaw (7) which carries them, forming two sides of an isosceles triangle.

5. Clamp for medical use according to claim 4, characterized by the fact
that the blades (10) have a length of about 3 centimeters and that the base of the triangle is of a length of about 2 centimeters.

6. Clamp for medical use according to any one of claims 1 to 5, characterized by the fact
that the gripping region (1) is formed by two legs (2a, 2b) with rings (3a, 3b) connected by a pivot forming the pivot of the jaws (7, 8), said jaws (7, 8) being each formed in prolongation of a leg (2a, 2b), on the side of the pivot (6) opposite the gripping region (1).

7. Multifunctional clamp for medical use according to claim 6 characterized by the fact
that the end (16a, 16b) of the legs (2a, 2b), located on the side of the pivot (6) opposite the gripping region (1), is incurved in a convex way and is off material resiliently deformable during pressure of the jaws (7, 8).

8. Multifunctional clamp for medical use according to claim 7 characterized by the fact that
the curvature of the two end portions (16a, 16b) is symmetrical.

9. Multifunctional clamp for medical use according to any one of claims 6 to 8, characterized by the fact
that the end portion (15a, 15b) of the legs (2a, 2b), located on the same side of the pivot (6) as the gripping region (1), is incurved concavely and is of a material that is resiliently deformable during pressure of the jaws (7, 8).

10. Multifunctional clamp for medical use according to claim 9 characterized by the fact
that the curvature of the two end portions (15a, 15b) is symmetrical.

11. Multifunctional clamp for medical use according to any one of claims 6 to 10, characterized by the fact
that the distance between the distal end of the jaws (7, 8) and the pivot (6) is 3 to 5 centimeters.

12. Multifunctional clamp for medical use according to any one of claims 1 to 5 characterized by the fact that it comprises:
two legs (2a, 2b) connected by a pivot (6) and each having at its end a ring (3a, 3b) to constitute the gripping region (1);
two secondary legs (20a, 20b) connected by a pivot (17) constituting the pivot of the jaws (7, 8), said jaws (7, 8) being each formed in prolongation of the end of a secondary leg (20a, 20b);
means for articulating the ends of the legs (2a, 2b) each with the end of one of the secondary legs (20a, 20b).

13. Multifunctional clamp for medical use according to claim 12 characterized by the fact that the articulation means comprise:
two symmetrical shells (19a, 19b) each secured to the end of a secondary leg (20a, 20b) and each provided with a hollow recess (22a, 22b) extending in the shell (19a, 19b) along the arc of a circle substantially perpendicular and centered relative to the axis (26) passing through the two pivots (6, 7), said hollow recesses (22a, 22b) being provided with an opening oriented opposite to the secondary leg (20a, 20b);
two hubs (18a, 18b) each secured to the end of one leg (2a, 2b) and disposed in the hollow recess (22a, 22b) of the shells (19a, 19b), the hubs (18a, 18b) being stopped in translation in their hollow recesses (22a, 22b).

14. Multifunctional clamp for medical use according to claim 12 characterized by the fact
that the articulation means comprise:
two symmetrical shells (19a, 19b) each secured to the end of a leg (2a, 2b) and each provided with a hollow recess (22a, 22b) extending into the shell (19a, 19b) along the arc of a circle substantially perpendicular to and centered relative to the axis (26) passing through the two pivots (6, 17) said hollow recesses (22a, 22b) being provided with an opening oriented opposite the leg (2a, 2b);
two hubs (18a, 18b) each secured to the end of a secondary branch (20a, 20b) and disposed in the hollow recess (22a, 22b) of the shells (19a, 19b), the hubs (18a, 18b) being stopped in translation in their hollow recess (22a, 22b).

15. Multifunctional clamp, for medical use according to claim 13 or 14 characterized by the fact that
the hubs (18a, 18b) are spherical,
the hollow recesses (22a, 22b) comprise cavities (23, 24) of shapes and dimensions complementary to those of the hubs (18a, 18b).
